(19) United States
(12) Patent Application Publication
(10) Pub. No.: US 2004/0247709 A1
(43) Pub. Date: Dec. 9, 2004

(54) Composition and method for treating upper abdominal pain and cramping

(75) Inventors: Dwight Mckee, Aptos, CA (US); Timothy A. Nolan, Batesville, AR (US)

Correspondence Address: WILLIAM J. SAPONE, COLEMAN SUDOL SAPONE P.C., 714 COLORADO AVENUE, BRIDGE PORT, CT, 06605, US

(73) Assignee: Pro-Health, Inc., Batesville, AR

(21) Appl. No.: 10/849,376

(22) Filed: May 19, 2004

Related U.S. Application Data
(60) Provisional application No. 60/476,818, filed on Jun. 6, 2003.

(51) Int. Cl. A61K 35/78; A61K 31/715

(52) U.S. Cl. 424/744; 514/561; 514/54; 514/563

(57) ABSTRACT

A composition for relieving the symptoms of upper intestinal tract discomfort contains aloe vera extract including acemannan, L-glutamine and L-glycine.

### CROSS-REFERENCE TO RELATED APPLICATION

This application claims priority in U.S. Provisional Patent Application Ser. No. 60/476,818 filed Jun. 6, 2003.

### TECHNICAL FIELD

This invention relates to a composition and method for treating stomach disorders and more particularly to a non-pharmaceutical dietary supplement for alleviating upper abdominal pain and cramping, as well as symptoms associated with inflammation of the lining of the upper intestinal tract.

### BACKGROUND

Upper intestinal discomfort can be caused by gastritis, acid reflux or duodenal inflammation, among other reasons. Such discomfort may be chronic or intermittent, and is common in many individuals. The symptoms of upper intestinal discomfort typically include upper abdominal pain, burning and/or cramping, often being more pronounced after meals.

Various treatments have been used to address the common causes, such as acid ablation for acid reflux. These treatments have a number of drawbacks. For example, acid ablation treatment involves administration of a proton pump inhibitor, such as Prilosec®, Prevacid®, Nexium®, etc. Proton pump inhibitors (“PPIs”) may cause a neutral upper gastro-intestinal tract pH that may enable bacterial and fungal growth. Acid ablation also interferes with digestion of proteins and absorption of vitamin B-12 as well as dietary minerals, and may result in muscle relaxation that can cause a significant increase in reflux once the treatment is stopped.

Such treatments while possibly necessary for chronic upper intestinal tract discomfort, generally should not be used by those suffering from more infrequent occurrences, or by those who wish to avoid use of medicinal treatments. Acid ablation is necessary to allow healing of erosive esophagitis, which if left untreated, may result in a significantly increased risk of malignancy of the gastro-esophageal junction. However, once healed, if alternative interventions can be introduced that will allow re-establishment of normal gastric pH, the disadvantages of chronic PPI therapy, as outlined above, can be avoided.

### SUMMARY OF THE INVENTION

It is an object of the present invention to provide a composition and method for treatment of the symptoms associated with upper intestinal discomfort that does not utilize acid ablation.

It is further object to provide a non-medicinal dietary/nutritional supplement to reduce or alleviate the symptoms associated with upper intestinal discomfort, specifically upper intestinal pain and cramping that may be associated with GI inflammation, as well as to support and stimulate the immune system, via the gut-associated lymphoid tissue (GALT).

It is a further object to provide a composition that is long acting in relieving the symptoms associated with upper intestinal discomfort.

These and other objects of the present invention are achieved by a composition for relieving or alleviating upper intestinal discomfort comprising aloe vera extract, L-glutamine, and L-glycine.

The method for alleviating the symptoms of upper intestinal discomfort comprises administering to a person in need of such treatment a composition containing aloe vera extract, L-glutamine and L-glycine, and a pharmaceutically acceptable carrier.

Using the three component mixture, which may be taken once or twice daily, such as in a drink, tablet, capsule, etc., provides complete relief of the symptoms of cramping and upper intestinal pain for as much as a full day. The result is achieved without any of the side effects typically associated with the medicinal treatments previously utilized, and thus provides a natural dietary supplement with significant therapeutic benefits.

### DETAILED DESCRIPTION OF THE INVENTION

Aloe is a plant known for its medicinal and therapeutic properties and various extracts are available from the plant. Among those useful in the present invention are the polysaccharide extracts and in particular, acemannan, a polysaccharide that is a long chain polymer of essentially linear beta 1-4 D-mannosyl units, specifically acetylated mannose molecules.

One aloe extract that is useful in the present invention is Manapol™ available from Carrington Laboratories, Inc. that contains roughly 25% acemannan, 25% pectin, 25% methyl cellulose and 25% calcium malate. Of course other aloe extracts that have therapeutically effective quantities of acemannan may also be used.

While aloe vera extracts are known for use in treating symptoms associated with irritable bowel syndrome, administration of aloe vera extract alone failed to relieve the symptoms of upper intestinal tract discomfort. It is believed that this is due to the difference in the cause of the symptoms as irritable bowel syndrome is associated with the lower intestinal tract, as opposed to the upper intestinal discomfort for which the present invention is targeted.

The second ingredient in the inventive composition is L-glutamine. L-glutamine is known for being useful as a source of fuel for cells lining the intestines. Glutamine also stimulates the synthesis of proteins, and is precursor for amino acids, proteins, glutathione and other biologically important molecules. It is also believed that L-glutamine can boost immune function and fight infection. While glutamine has been used for treating ulcers due to the ability to support and build the intestinal lining, there has been no suggestion of use in treating generalized upper intestinal discomfort.

A combination of glutamine and aloe vera extract was given to an individual who had upper intestinal tract discomfort. The individual was given 80 mg. of Manapol and 1 gram of L-glutamine. While the upper abdominal pain resolved completely within 15 minutes, the relief was of limited duration and the discomfort returned in full force within a few hours.

The third ingredient that is used in the inventive composition is L-glycine. Glycine is classified as a non-essential amino acid, though it is considered conditionally essential for synthesis of nucleic acids, bile acids and other non-essential amino acids. It has also been used in gastric antacids as it contributes to the acid neutralizing effect.

It has been surprisingly discovered that the combination of aloe vera extract, glutamine and glycine is particularly effective in providing long term relief of upper intestinal tract discomfort.

A person suffering from upper intestinal tract discomfort was given eight ounces of the inventive formulation comprising aloe vera extract containing 320 mg Manapol™, 250 mg. of L-glycine and 1 gram of glutamine. The person reported complete relief for an entire day. The next day only the aloe extract and L-glutamine were administered, and again the relief was of short term duration. Return to the three component mixture brought repeated complete daily relief. Only with the combination of all three ingredients was complete all day duration relief achieved.

Utilizing the present invention, upper intestinal pain and cramping, associated with heartburn, acid reflux, inflammation or another cause, are substantially alleviated by the daily administration of the combination of aloe vera extract, 1-glutamine and 1-glycine.

The aloe vera extract preferably contains acemannan and is given at from about 30-300 mg., more preferably about 50-150 mg. and most preferably 60 mg. per day. This may be adjusted to account for the proportion of acemannan in the extract. For example, as Manapol contains about 25% wt. acemannan, then 40 mg. of aloe vera extract would contain 10 mg. acemannan, and the ranges described above may be adjusted to allow use of other aloe vera extracts while still providing comparable quantities of acemannan in the inventive formulation. In a preferred embodiment, the composition is administered in a tableted formulation containing 30 mg aloe vera extract, such as Manapol (approx. 7.5 mg acemannan), 250 mg L-glycine, and 300 mg L-glutamine. Administration can be of two tablets together, though use of one tablet twice daily is preferred.

The L-glutamine may be administered at from about 200-5000 mg. per day, more preferably 500-1500 milligrams, and most preferably 600 milligrams per day.

The L-glycine may be administered at from about 100-800 mg. per day, more preferably 200-700 mg. per day, most preferably 500 mg. per day.

While the three ingredient combination is necessary to achieve the results of the present invention, it is possible to include other ingredients that provide beneficial effects without degrading the effectiveness of the combination. Those may include but are not limited to various vitamin, mineral and herbal supplements, antioxidants, amino acids, etc. Suitable carriers for delivering the composition as a prefabricated drink, powdered drink mix, granules, powders, coated tablets, caplet, hard or elastic capsules, coated tablets, tablet, soft gel liquid, emulsion, suspension, syrup or other form may also be used and any routes of administration typical for the administration of dietary and nutritional supplements can easily be adapted for delivery of the inventive composition. It should also be understood that various other formulating ingredients such as thickeners, flavoring agents, etc. as are necessary for formulating the inventive composition may also be used.

Adjuvants normally used in formulating medicaments in the above-exemplified forms may equally be used as pharmaceutically acceptable liquid or solid diluents or carriers for formulating the compositions of this invention. Specific Examples include syrup, gum Arabic, gelatin, sorbitol, tragacanth, polyvinyl pyrrolidone, magnesium stearate, talc, polyethylene glycol, silica, lactose, sucrose, corn starch, calcium phosphate, glycine, potato starch, carboxymethyl cellulose calcium, sodium laurylsulfate, water, ethanol, glycerol, mannitol, and a phosphate buffer, among others.

The inventive composition, if required, may further contain other adjuvants customarily used in the field of pharmaceutical or nutraceutical formulation, such as coloring agents, flavors, corrigents, antiseptics, dissolution acids, suspending agents and dispersing agents.

Liquids administered orally may include flavoring agents such as mint, cherry, guava, citrus, cinnamon, orange, mango, or mixed fruit flavors. Pills, capsules or tables administered orally may also include flavoring agents. Additionally, all compositions may further comprise agents to increase shelf-life, such as preservatives, anti-oxidants and other components necessary and suitable for manufacture and distribution of the composition.

In a preferred embodiment of the present invention, a 580 mg tablet, containing 30 mg of an aloe vera extract such as Manapol, 300 mg glutamine, and 250 mg glycine, as well as microcrystalline cellulose, silica and magnesium stearate, is given on a twice daily.

It is believed that the long term use of the combination will not only alleviate the symptoms associated with upper intestinal discomfort but may also serve to decrease intestinal permeability and promote healing of the intestine, without the side effects associated with prior medicinal treatments that may significantly alter acidity thereby avoiding the potential for bacterial growth or fungal growth in the intestine. Small intestine hyperpermeability is a common occurrence in modern populations, due to a variety of factors, including the frequent use of aspirin and other non-steroidal anti-inflammatory drugs (NSAIDS). These medications block the production of prostaglandins. Prostaglandin E1 plays an essential role in maintaining the semi-permeable barrier of the small intestine. When this barrier is compromised, larger molecules than would normally be absorbed, such as incompletely digested dietary proteins and peptides can be absorbed intact, and may result in an immune response to the molecule, which may be interpreted by the immune system as a foreign substance, rather than as a nutrient.

The condition of increased permeability of the small intestine is not associated with any specific symptoms that indicate its occurrence, nor can it be diagnosed by appearance of the intestinal lining at endoscopy. It can, however be tested for by the differential absorption of mannitol (a simple sugar that should be well absorbed) and lactulose (a large sugar molecule that should not be well absorbed by the healthy small intestine). Ingesting a standardized solution of these two sugars, followed by urine collection and laboratory assay of the mannitol and lactulose content is a reliable test for presence of either normal or increased permeability of the small intestine.

Many experts currently believe that increased intestinal permeability underlies the development of a wide variety of symptoms of food sensitivity and intolerance, such as headache, bloating, fatigue, irritability, inability to think clearly, and joint pain, among others. Because of this mechanism, it is believed that long term use of the invention may have a positive impact on a wide variety of health problems related to food sensitivity and intolerance.

The inventive formulation may also be formulated as a lozenge for slow release in the mouth for the treatment of oral inflammation and pain, such as from canker sores, mouth sores, etc., and to assist in healing inflammation in the oral cavity.

While preferred embodiments of the present invention have been shown and described, it will be understood by those skilled in the art that various changes or modifications can be made without varying from the scope of the present invention.

What is claimed is:

1. A composition for alleviating the symptoms of upper intestinal discomfort such as pain and cramping comprising aloe vera extract including acemannan, 1-glutamine and 1-glycine.

2. The composition of claim 1 wherein the aloe vera extract is present at from about 30-300 mg., when the extract contains about 25% acemannan.

3. The composition of claim 1 wherein the aloe vera extract is present at from about 50-150 mg. when the extract contains about 25% accemannan.

4. The composition of claim 1 wherein the aloe vera extract is present at about 60 mg.

5. The composition of claim 1 wherein the L-glutamine is present at from about 200-5000 mg.

6. The composition of claim 1 wherein the L-glutamine is present at from about 500-1500 mg.

7. The composition of claim 1 wherein the L-glutamine is present at about 600 mg.

8. The composition of claim 1 further comprising a pharmaceutically acceptable carrier.

9. The composition of claim 1 wherein the L-glycine is present at from about 100-800 mg.

10. The composition of claim 1 wherein the L-glycine is present at from about 200-700 mg.

11. The composition of claim 1 wherein the L-glycine is present at about 500 mg.

12. The composition of claim 1 wherein the composition is in a unit or multidose form.

13. The composition of claim 1 further comprising at least one ingredient from the group consisting of vitamin, mineral and herbal supplements, antioxidants, amino acids, thickeners, flavoring agents, and coloring agents.

14. A method for alleviating the symptoms of upper intestinal tract discomfort comprising administering to a person in need of such treatment a composition containing aloe vera extract including acemannan, 1-glutamine and 1-glycine.

15. The method of claim 14 wherein the composition further comprises a pharmaceutically acceptable carrier.

16. The method of claim 14 wherein the composition is administered in unit or multiple dosage form.

17. The method of claim 14 wherein the aloe vera extract is present at from about 30-300 mg., when the extract contains about 25% acemannan.

18. The method of claim 14 wherein the aloe vera extract is present at from about 50-150 mg. when the extract contains about 25% accemannan.

19. The method of claim 14 wherein the aloe vera extract is present at about 60 mg.

20. The method of claim 14 wherein the L-glutamine is present at from about 200-5000 mg.

21. The method of claim 14 wherein the L-glutamine is present at from about 500-1500 mg.

22. The method of claim 14 wherein the L-glutamine is present at about 600 mg.

23. The method of claim 14 wherein the L-glycine is present at from about 100-800 mg.

24. The method of claim 14 wherein the L-glycine is present at from about 200-700 mg.

25. The method of claim 14 wherein the L-glycine is present at about 500 mg.

26. The method of claim 14 wherein the composition is administered orally.

27. The method of claim 14 further comprising at least one ingredient from the group consisting of vitamin, mineral and herbal supplements, antioxidants, amino acids, thickeners, flavoring agents, and coloring agents.
